(19) United States
(12) Patent Application Publication
(10) Pub. No.: US 2019/0136187 A1
(43) Pub. Date: May 9, 2019

(54) Blood Isolation and Extraction Method and Device Thereof

(71) Applicants: National University of Kaohsiung, Kaohsiung (TW); Biomimedtech Co., Ltd., Kaohsiung (TW); Neoasia Limited, Taipei City (TW)

(72) Inventors: Yi-Chang Chung, Kaohsiung (TW); Kai-Ming Wu, Kaohsiung (TW); Che-Wen Chang, Kaohsiung (TW)

(21) Appl. No.: 16/236,677

(22) Filed: Dec. 31, 2018

Related U.S. Application Data
(63) Continuation-in-part of application No. 15/193,083, filed on Jun. 26, 2016.

(51) Int. Cl. C12N 5/078; B01D 39/08; B01D 39/16

(52) U.S. Cl. CPC C12N 5/0644; B01D 2239/0421; B01D 39/16; B01D 39/083

(57) ABSTRACT

A blood isolation and extraction method includes: providing a predetermined amount of blood; utilizing a platelet filter unit to filter the predetermined amount of blood to generate a filtered blood; utilizing a plasma separation unit to divide the filtered blood into a plasma layer and a blood cell layer for separating blood cells from blood plasma; and extracting the blood plasma from the plasma layer and the blood cells from the blood cell layer. In another embodiment, the blood isolation and extraction method further includes: providing a platelet-washing unit to wash the platelet filter unit with a solution to produce a platelet solution; and mixing the platelet solution with the blood plasma to produce a platelet and plasma mixed solution.

### CROSS REFERENCE TO RELATED APPLICATIONS

This application is a continuation-in-part of U.S. patent application Ser. No. 15/193,083, filed Jun. 26, 2016, which is hereby incorporated by reference in its entirety.

### BACKGROUND OF THE INVENTION

### 1. Field of the Invention

The present invention relates to a blood isolation and extraction method and device thereof. Particularly, the present invention relates to the blood isolation and extraction method and device thereof for separately extracting platelets, plasma and corpuscles.

### 2. Description of the Related Art

By way of example, Taiwanese Patent No. 1395612, entitled “BLOOD SEPARATION METHOD,” discloses a conventional blood separation method. The conventional blood separation method includes the steps of: providing a filtering film having a flow channel and a plurality of holes and a receiving compartment connecting with the filtering film and communicating with the flow channel; actuating a flow of blood through the filtering film to substantially form a horizontal movement with respect to the filtering film; and collecting the blood passing through the holes in the receiving compartment. The filtering film can generate a shear stress to block blood cells (i.e., hemocyte) so that the rest of blood (i.e., plasma) passes through the holes of the filtering film.

The filtering film has a first surface provided with a plurality of cambered surfaces and a second surface provided with a plurality of recessions. On the first surface, each of the holes is formed among the cambered surfaces and is aligned with each of the recessions provided on the second surface. The holes have a diameter ranging from 1-50 micrometers. The filtering film is made of a material of metal or metal alloy.

However, the filtering film applied in the blood separation method is only suitable for roughly separating blood cells with the 1-50 micrometer holes and cannot successfully separate platelets from blood cells (i.e., red blood cells or white blood cells). Hence, there is a need of providing an improved blood separation method and system for extracting platelets from blood cells.

U.S. Pat. No. 6,893,412, entitled “PLATELET COLLECTING APPARATUS,” discloses a conventional platelet collecting apparatus. The platelet collecting apparatus comprises a centrifugal separator possessing a rotatable rotor, a first line for allowing the flow of the blood entering the centrifugal separator, a second line for allowing the flow of the blood emanating from the centrifugal separator, and a plasma collecting bag connected to the first line and the second line to collect the plasma emanating from the centrifugal separator and to return the collected plasma to the centrifugal separator.

The platelet collecting apparatus further comprises a platelet collecting bag connected to the second line to collect the platelets emanating from the centrifugal separator, a blood delivering pump disposed in the first line, and a controller for controlling the operation of the rotor of the centrifugal separator and the operation of the blood delivering pump. The controller is provided with a function of varying the rotational frequency of the rotor during the course of blood collection in conformity with the amount of the blood entering into the centrifugal separator via the first line.

The platelet collecting apparatus must utilize the centrifugal separator to separate platelets from plasma in a centrifugal separation manner. However, the centrifugal separation manner results in lengthening a total processing time of blood separation and in increasing a total cost and a total weight of blood separation apparatus. Hence, there is a need of providing an improved blood separation method and system for extracting platelets from blood cells. The above-mentioned patents are incorporated herein by reference for purposes including, but not limited to, indicating the background of the present invention and illustrating the situation of the art.

As is described in greater detail below, the present invention provides a blood isolation and extraction method and device thereof. A platelet filter unit is provided to filter a predetermined amount of blood to generate a filtered blood. The platelet filter unit is washed with a solution to produce a platelet solution which contains platelets remaining on the platelet filter unit. A plasma separation unit is provided to divide the filtered blood into a plasma layer and a blood cell layer for separating blood cells from blood plasma in such a way to improve the conventional blood separation method and device.

### SUMMARY OF THE INVENTION

The primary objective of this invention is to provide a blood isolation and extraction method. A platelet filter unit is provided to filter a predetermined amount of blood to generate a filtered blood. The platelet filter unit is washed with a solution to produce a platelet solution which contains platelets remaining on the platelet filter unit. A plasma separation unit is provided to divide the filtered blood into a plasma layer and a blood cell layer for separating blood cells from blood plasma. Advantageously, the blood isolation and extraction method of the present invention is successful in rapidly separating the platelets and blood cells from the blood plasma.

The blood isolation and extraction method in accordance with an aspect of the present invention includes:

providing a predetermined amount of blood;

utilizing a platelet filter unit to filter the predetermined amount of blood to generate a filtered blood;

utilizing a plasma separation unit to divide the filtered blood into a plasma layer and a blood cell layer for separating blood cells from blood plasma; and

extracting the blood plasma from the plasma layer and the blood cells from the blood cell layer.

The blood isolation and extraction method in accordance with another aspect of the present invention includes:

providing a predetermined amount of blood;

utilizing a platelet filter unit to filter the predetermined amount of blood to generate a filtered blood;

washing or flushing the platelet filter unit with a solution to produce a platelet solution which contains platelets remaining on the platelet filter unit;

utilizing a plasma separation unit to divide the filtered blood into a plasma layer and a blood cell layer for separating blood cells from blood plasma; and

mixing the platelet solution with the blood plasma retrieved from the plasma layer to produce a platelet and plasma mixed solution.

In a separate aspect of the present invention, the predetermined amount of blood is supplied by a blood supply unit or an injector.

In a further separate aspect of the present invention, the platelet filter unit includes a platelet adsorption net filter or a platelet adsorption plate.

In yet a further separate aspect of the present invention, the platelet adsorbing net filter includes a plurality of apertures and is made of platelet adsorption resin.

In yet a further separate aspect of the present invention, the platelet adsorbing plate is made of platelet adsorption resin.

In yet a further separate aspect of the present invention, the plasma separation unit includes a blood cell adsorbent, a blood cell adsorption material or a blood cell adsorption gel.

In yet a further separate aspect of the present invention, the plasma separation unit includes a tube to contain the plasma layer and the blood cell layer formed in the filtered blood.

Another objective of this invention is to provide a blood isolation and extraction device. A platelet filter unit is provided to filter a predetermined amount of blood to generate a filtered blood. The platelet filter unit is washed with a solution to produce a platelet solution which contains platelets remaining on the platelet filter unit. A plasma separation unit is provided to divide the filtered blood into a plasma layer and a blood cell layer for separating blood cells from blood plasma. Advantageously, the blood isolation and extraction device of the present invention is successful in rapidly separating the platelets and blood cells from the blood plasma.

The blood isolation and extraction device in accordance with an aspect of the present invention includes:

a platelet filter unit provided to filter a predetermined amount of blood to generate a filtered blood; and

a plasma separation unit provided to divide the filtered blood into a plasma layer and a blood cell layer for separating blood cells from blood plasma;

wherein the blood plasma is retrieved from the plasma layer and the blood cells are retrieved from the blood cell layer.

The blood isolation and extraction device in accordance with another aspect of the present invention includes:

a platelet filter unit provided to filter a predetermined amount of blood to generate a filtered blood;

a platelet-washing unit provided to wash or to flush the platelet filter unit with a solution to produce a platelet solution which contains platelets remaining on the platelet filter unit; and

a plasma separation unit provided to divide the filtered blood into a plasma layer and a blood cell layer for separating blood cells from blood plasma;

wherein the blood plasma is retrieved from the plasma layer and is further mixed with the platelet solution to produce a platelet and plasma mixed solution.

In a separate aspect of the present invention, the predetermined amount of blood is supplied by a blood supply unit or an injector.

In a further separate aspect of the present invention, the platelet filter unit includes a platelet adsorption net filter or a platelet adsorption plate.

In yet a further separate aspect of the present invention, the platelet adsorbing net filter includes a plurality of apertures and is made of platelet adsorption resin.

In yet a further separate aspect of the present invention, the platelet adsorbing plate is made of platelet adsorption resin.

In yet a further separate aspect of the present invention, the plasma separation unit includes a blood cell adsorbent, a blood cell adsorption material or a blood cell adsorption gel.

In yet a further separate aspect of the present invention, the plasma separation unit includes a tube to contain the plasma layer and the blood cell layer formed in the filtered blood.

Further scope of the applicability of the present invention will become apparent from the detailed description given hereinafter. However, it should be understood that the detailed description and specific examples, while indicating preferred embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a flowchart of a blood isolation and extraction method in accordance with a first preferred embodiment of the present invention.

FIG. 2 is a schematic block diagram of a blood isolation and extraction device in accordance with a first preferred embodiment of the present invention.

FIG. 2A is a schematic block diagram of a blood-cell agglutination separation device in accordance with another preferred embodiment of the present invention.

FIG. 3 is a flowchart of a blood isolation and extraction method in accordance with a second preferred embodiment of the present invention.

FIG. 4 is a schematic block diagram of a blood isolation and extraction device in accordance with a second preferred embodiment of the present invention.

FIG. 5 is a schematic block diagram of a blood isolation and extraction device in accordance with a third preferred embodiment of the present invention.

FIG. 6 is a schematic block diagram of a blood-cell agglutination separation device in accordance with a fourth preferred embodiment of the present invention.

FIG. 7 is a flowchart of a blood-cell agglutination separation method in accordance with another preferred embodiment of the present invention.

FIG. 8 is a schematic block diagram of a blood-cell agglutination separation device in accordance with a fifth preferred embodiment of the present invention.

FIG. 9 is a schematic block diagram of a blood-cell agglutination separation device in accordance with a sixth preferred embodiment of the present invention.

FIGS. 10(a)-10(g) is a set of chemical structure views of a series of amino monomers applied in a blood-component adhesion material in accordance with a preferred embodiment of the present invention.

FIG. 11 is a chemical structure view of monomer having a hydrophobic group applied in the blood-component adhesion material in accordance with another preferred embodiment of the present invention.

### DETAILED DESCRIPTION OF THE INVENTION

It is noted that a blood isolation and extraction method and device thereof in accordance with the preferred embodiment of the present invention is suitable for manufacturing various whole blood products and related products thereof, including packed blood cells, fresh frozen plasma, cryoprecipitate, platelet concentrate, leukocyte concentrate and related products thereof for example, which are not limitative of the present invention. The blood isolation and extraction method and device thereof in accordance with the preferred embodiment of the present invention can also be applicable to various medical research, medical treatment (e.g. Achilles tendon rupture or arthritis treatment), medical cosmetology (e.g. baldness, wrinkle or collagen treatment), medical rehabilitation (e.g. plastic surgery or dental implant surgery), pharmaceutical products or related industries thereof, which are not limitative of the present invention.

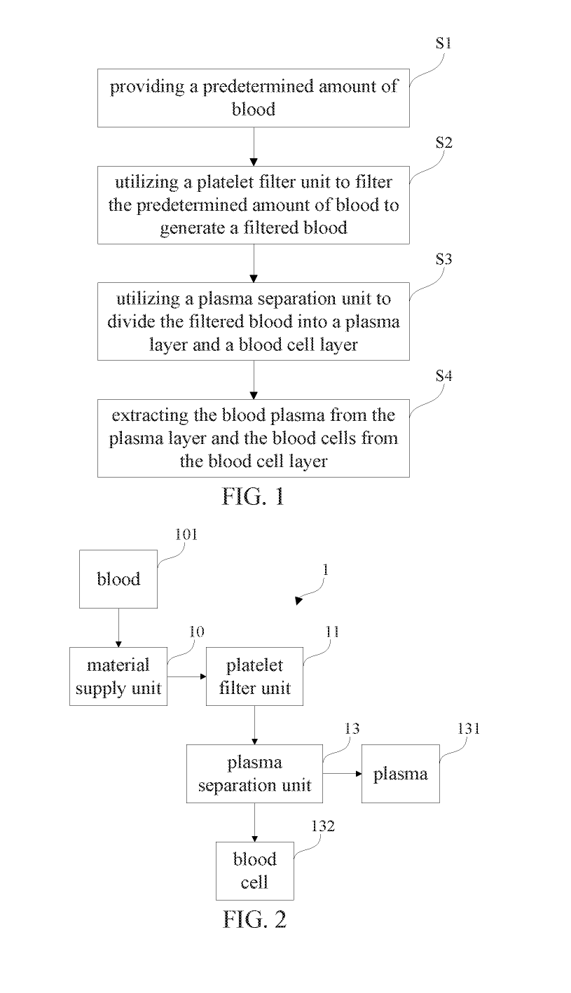

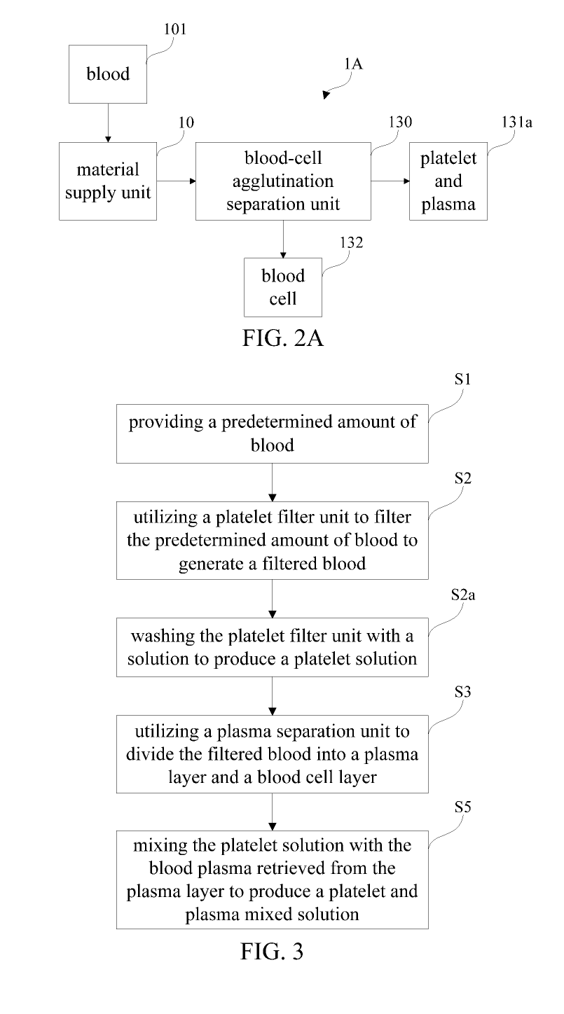

FIG. 1 shows a flowchart of a blood isolation and extraction method in accordance with a first preferred embodiment of the present invention. FIG. 2 shows a schematic block diagram of a blood isolation and extraction device in accordance with the first preferred embodiment of the present invention, corresponding to the blood isolation and extraction method shown in FIG. 1. Referring now to FIGS. 1 and 2, the blood isolation and extraction device 1 in accordance with the first preferred embodiment of the present invention includes a material supply unit 10 (e.g. pump, injector or the like), a platelet filter unit 11 (e.g. platelet filter device) and a plasma separation unit 13 (e.g. plasma separation device) which can meet gamma radiation sterilization and biocompatibility requirement.

Still referring to FIGS. 1 and 2, by way of example, the material supply unit 10 connects the platelet filter unit 11 with a first conduit or pipeline and the platelet filter unit 11 further connects the plasma separation unit 13 with a second conduit or pipeline. The material supply unit 10 is suitably installed with a blood-drawing unit 101 or other blood-collecting devices (e.g., venipuncture syringes or test tubes).

FIG. 2A shows a schematic block diagram of a blood-cell agglutination separation device in accordance with another preferred embodiment of the present invention. Referring to FIGS. 2 and 2A, in comparison with the first embodiment, the blood-cell agglutination separation device 1A in accordance with the preferred embodiment of the present invention omits the platelet filter unit 11 for simplifying the entire structure. The blood-cell agglutination separation device 1A includes a material supply unit 10 (e.g. pump, injector or the like) and a blood-cell agglutination separation unit 130. The material supply unit 10 directly connects the blood-cell agglutination separation unit 130 with a conduit or pipeline for supplying a predetermined amount of blood or whole blood. The blood-cell agglutination separation unit 130 contains a predetermined amount of blood-component adhesion materials for accelerating a separation of blood plasma and blood corpuscles (i.e. blood cells). However, the blood-component adhesion material can attract blood plasmas and proteins to cause unbalance of components (i.e. blood cells and plasma) of blood or whole blood such that heavy blood cells (i.e. white blood cells and red blood cells) can be automatically precipitated in the static blood due to the gravitational force.

With continued to FIGS. 1 and 2, the blood isolation and extraction method in accordance with the first preferred embodiment of the present invention includes the step S1: automatically, semi-automatically or manually operating the material supply unit 10 to supply a predetermined amount of blood (i.e. whole blood) to the platelet filter unit 11. By way of example, the material supply unit 10 includes an injector, a syringe or a pump device to operate the blood-drawing unit 101 so as to supply the blood to the platelet filter unit 11. In addition, the material supply unit 10 further includes a server motor or the like to pump the blood.

With continued to reference FIGS. 1 and 2, the blood isolation and extraction method in accordance with the first preferred embodiment of the present invention includes the step S2: automatically, semi-automatically or manually operating the platelet filter unit 11 to filter the predetermined amount of blood to generate a filtered blood. By way of example, after the predetermined amount of blood is pumped to pass through the platelet filter unit 11 with a predetermined pressure, platelets are selectively adsorbed and remaining on a surface portion of the platelet filter unit 11. Thereafter, the platelets will not be contained in the filtered blood. Advantageously, the blood isolation and extraction method of the present invention can avoid problems of rapid loss of filtered blood pressure, velocity drop of filtered blood flow and choke of filter holes in filtering operation.

With continued to reference FIGS. 1 and 2, by way of example, the platelet filter unit 11 includes a platelet adsorption net filter or a platelet adsorption plate. The platelet adsorbing net filter includes a plurality of apertures performed as filtering holes and is made of platelet adsorption resin or other equivalent materials. The platelet adsorption plate is also made of platelet adsorption resin other equivalent materials. The material of platelet adsorption resin has a function of platelet adsorption and a high degree of recovery of platelets.

By way of example, the platelet adsorption resin or resin is modified by a surface modifier selected from modifiers containing phosphatidyl choline (pc) derivatives or zwitterionic dopamine derivatives which have highly stable and water absorbability. The zwitterionic structure can avoid bonding with other metal bonds, polynucleotide and proteins. In addition, the zwitterionic structure has a high degree of hydrophile even though it can absorb four times of water to contain proteins after dried. Advantageously, it is hard to be permeated or absorbed to prevent clot reaction or hemolytic reaction.

With continued to reference FIGS. 1 and 2, the blood isolation and extraction method in accordance with the first preferred embodiment of the present invention includes the step S3: transferring the filtered blood to the plasma separation unit 13 and automatically, semi-automatically or manually operating the plasma separation unit 13 to divide the filtered blood into a plasma layer and a blood cell layer in a predetermined tube for separating blood cells (i.e. corpuscles) from blood plasma. Generally, the plasma layer is an upper layer (i.e. creamy yellow) relatively while the blood cell layer is a lower layer.

With continued to reference FIGS. 1 and 2, by way of example, the plasma separation unit 13 includes a blood cell adsorbent, a blood cell adsorption gel or other blood cell adsorption materials. The blood cell adsorbent or the blood cell adsorption gel is cured on sponges, foam rubber. hemostatic cotton or surfaces of other fillers which are preferably contained in a cone container.

With continued to reference FIGS. 1 and 2, the blood isolation and extraction method in accordance with the first preferred embodiment of the present invention includes the step S4: automatically, semi-automatically or manually operating a tool (e.g. pipette or other equivalent tools) to draw out the liquids of plasma layer and blood cell layer for extracting the blood plasma from the plasma layer and the blood cells from the blood cell layer.

With continued to reference FIGS. 1 and 2, by way of example, the blood plasma is stored in a plasma storage unit 131 (i.e. first sterilized container or other equivalent devices) while the blood cells are separately stored in a blood cell storage unit 132 (i.e. second sterilized container or other equivalent devices).

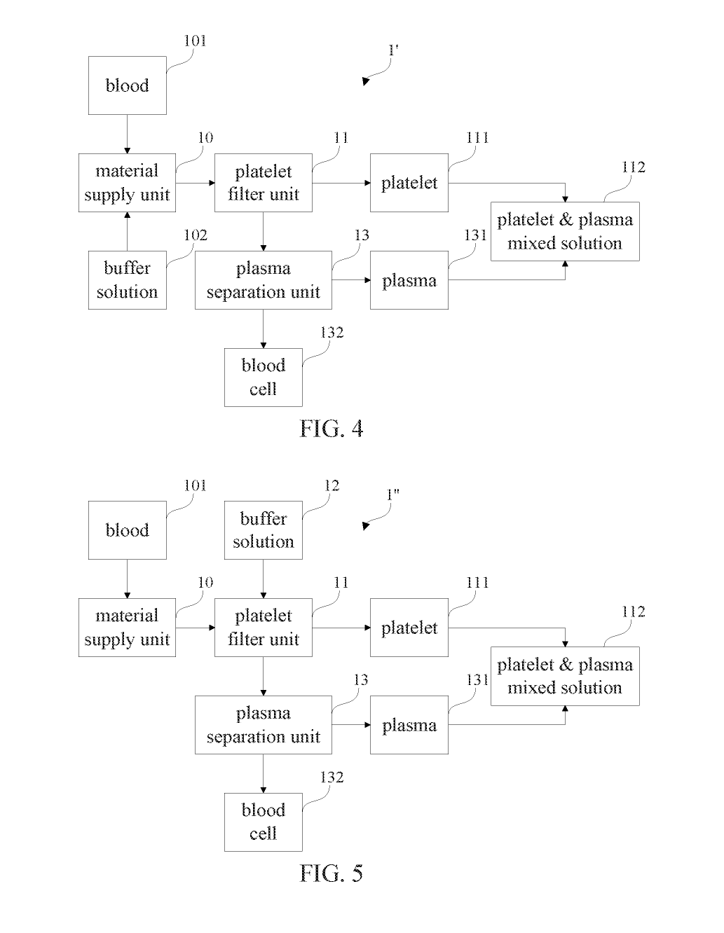

FIG. 3 shows a flowchart of a blood isolation and extraction method in accordance with a second preferred embodiment of the present invention. FIG. 4 shows a schematic block diagram of a blood isolation and extraction device in accordance with the second preferred embodiment of the present invention, corresponding to the blood isolation and extraction method shown in FIG. 3.

Referring now to FIGS. 3 and 4, the blood isolation and extraction device 1′ in accordance with the second preferred embodiment of the present invention includes a material supply unit 10, a blood-drawing unit 101, a buffer supply unit 102, a platelet filter unit 11 and a plasma separation unit 13 which can meet gamma radiation sterilization and biocompatibility requirement.

Still referring to FIGS. 3 and 4, by way of example, the blood-drawing unit 101 and the buffer supply unit 102 connect the material supply unit 10 with a first conduit, the material supply unit 10 further connects the platelet filter unit 11 with a second conduit and the platelet filter unit 11 further connects the plasma separation unit 13 with a third conduit. The material supply unit 10 is suitably installed with a blood supply source or other blood-collecting devices.

With continued to FIGS. 3 and 4, the blood isolation and extraction method in accordance with the second preferred embodiment of the present invention includes the step S1: automatically, semi-automatically or manually operating the material supply unit 10 to supply a predetermined amount of blood (i.e. whole blood) to the platelet filter unit 11. By way of example, the material supply unit 10 includes an injector, a syringe or a pump device to operate the blood-drawing unit 101 so as to supply the blood to the platelet filter unit 11. In addition, the material supply unit 10 further includes a server motor or the like to pump the blood.

With continued to reference FIGS. 3 and 4, the blood isolation and extraction method in accordance with the second preferred embodiment of the present invention includes the step S2: automatically, semi-automatically or manually operating the platelet filter unit 11 to filter the predetermined amount of blood to generate a filtered blood. By way of example, after the predetermined amount of blood is pumped to pass through the platelet filter unit 11 with a predetermined pressure, platelets are selectively adsorbed and remaining on a surface portion of the platelet filter unit 11. Thereafter, the platelets will not be contained in the filtered blood. Advantageously, the blood isolation and extraction method of the present invention can avoid problems of rapid loss of filtered blood pressure, velocity drop of filtered blood flow and choke of filter holes in filtering operation.

With continued to reference FIGS. 3 and 4, the blood isolation and extraction method in accordance with the second preferred embodiment of the present invention includes the step S2a: automatically, semi-automatically or manually operating the material supply unit 10 and the buffer supply unit 102 to wash or to flush the platelet filter unit 11 with a solution (or a buffer solution) at least one or several times to produce a platelet solution which contains platelets remaining on the platelet filter unit 11. By way of example, the platelet solution is stored in a platelet storage unit 111 (i.e. sterilized container). In platelet washing operation, the material supply unit 10 has ceased to supply the blood from the blood-drawing unit 101.

With continued to reference FIGS. 3 and 4, by way of example, the material supply unit 10 includes an injector, a syringe or a pump device to operate the buffer supply unit 102 to form a platelet-washing unit which can wash or flush the platelets on the platelet filter unit 11 with the buffer solution (e.g. phosphate buffer solution, PBS).

With continued to reference FIGS. 3 and 4, the blood isolation and extraction method in accordance with the second preferred embodiment of the present invention includes the step S3: transferring the filtered blood to the plasma separation unit 13 and automatically, semi-automatically or manually operating the plasma separation unit 13 to divide the filtered blood into a plasma layer and a blood cell layer in a predetermined tube for separating blood cells from blood plasma. Generally, the plasma layer is an upper layer (i.e. creamy yellow) relatively while the blood cell layer is a lower layer.

With continued to reference FIGS. 3 and 4, the blood isolation and extraction method in accordance with the second preferred embodiment of the present invention includes the step S5: automatically, semi-automatically or manually mixing the platelet solution with the blood plasma retrieved from the plasma layer to produce a platelet and plasma mixed solution. By way of example, the platelet and plasma mixed solution is stored in a mixed solution storage unit 112 (i.e. sterilized container).

FIG. 5 shows a schematic block diagram of a blood isolation and extraction device in accordance with a third preferred embodiment of the present invention. Referring to FIG. 5, the blood isolation and extraction device 1″ in accordance with the second preferred embodiment of the present invention includes a material supply unit 10, a blood-drawing unit 101, a platelet filter unit 11, a platelet-washing unit 12 and a plasma separation unit 13 which can meet gamma radiation sterilization and biocompatibility requirement.

Still referring to FIG. 5, by way of example, the platelet-washing unit 12 connects the platelet filter unit 11 with a conduit. In platelet washing operation, the platelet-washing unit 12 is automatically, semi-automatically or manually operated to wash or to flush the platelet filter unit 11 with a solution (or a buffer solution) at least one or several times to produce a platelet solution. Synchronously, the material supply unit 10 has ceased to supply the blood from the blood-drawing unit 101.

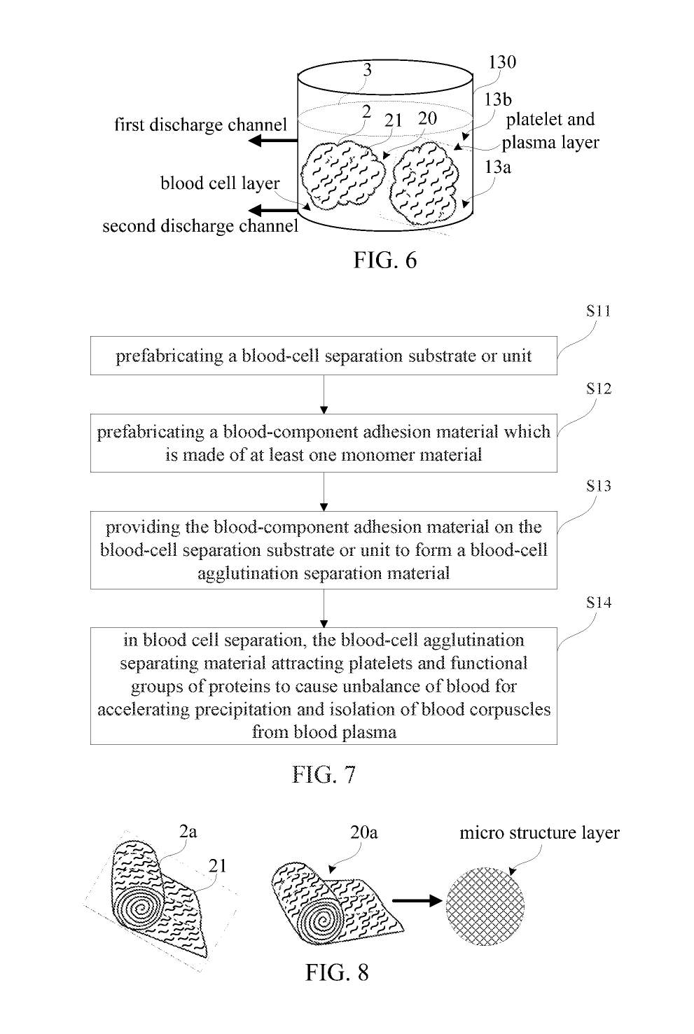

FIG. 6 shows a schematic block diagram of a blood-cell agglutination separation device in accordance with a fourth preferred embodiment of the present invention. Referring now to FIG. 6, the blood-cell agglutination separation device in accordance with the fourth preferred embodiment of the present invention includes a blood-cell agglutination separation unit 130 and a blood-cell agglutination separation material 20. According to different needs of blood qualities (e.g. platelet-rich plasma (PRP), leukocyte-rich PRP or leukocyte-poor PRP), various sizes of blood-cell agglutination separation unit 130 and various amounts of blood-cell agglutination separation material 20 are provided.

With continued reference to FIG. 6, the blood-cell agglutination separation unit 130 contains a predetermined amount of untreated blood 3 and has a blood-cell agglutination area 13a and a platelet-rich plasma area 13b. In a preferred embodiment, the blood-cell agglutination separation unit 130 is formed from a separation bottle device, a blood-passage separation bottle device or a negative pressure vessel device.

Still referring to FIG. 6, a syringe device or a blood transfusion device can supply the predetermined amount of untreated blood 3 to the blood-cell agglutination separation unit 130. In another preferred embodiment, the syringe device or the blood transfusion device is fixed on the blood-cell agglutination separation unit 130 or is detachably separated from the blood-cell agglutination separation unit 130.

FIG. 7 shows a flowchart of a blood-cell agglutination separation method in accordance with another preferred embodiment of the present invention. Referring to FIGS. 6 and 7, the blood-cell agglutination separation method in accordance with the preferred embodiment of the present invention includes the step S11: prefabricating a blood-cell separation substrate or unit 2 (e.g. cotton ball), as best shown in FIG. 6. By way of example, the blood-cell separation substrate or unit 2 is made of a cotton material, a gauze material, a bandage material or a combination thereof (e.g. cotton-core gauze material or bandage wrap roll material).

With continued reference to FIGS. 6 and 7, the blood-cell agglutination separation method in accordance with the preferred embodiment of the present invention includes the step S12: prefabricating a blood-component adhesion material 21 which is made of at least one monomer material. By way of example, the monomer material is selected from a platelet-adhesion monomer material, a protein-adhesion monomer material, a monomer material having a hydrophobic functional group, a platelet-adhesion and platelet-releasing monomer material.

With continued reference to FIGS. 6 and 7, the blood-cell agglutination separation method in accordance with the preferred embodiment of the present invention includes the step S13: automatically, semi-automatically or manually providing a predetermined amount of the blood-component adhesion material 21 on the blood-cell separation substrate or unit 2 to form a blood-cell agglutination separation material 20.

With continued reference to FIGS. 6 and 7, the blood-component adhesion material 21 can be selectively dispensed on a predetermined position of the blood-cell separation substrate or unit 2 by polymerization, graft copolymerization, chemical deposition or coating. The predetermined position of the blood-cell separation substrate or unit 2 includes a central area, a peripheral edge area, a corner area or other suitable areas.

With continued reference to FIGS. 6 and 7, the blood-cell agglutination separation method in accordance with the preferred embodiment of the present invention includes the step S14: in blood cell separation for a predetermined separating time (e.g. 20 or 30 minutes), the blood-cell agglutination separation material 20 attracting platelets and functional groups of blood proteins to thereby cause unbalance of blood for automatically accelerating precipitation and isolation of blood corpuscles from blood plasma in the blood-cell agglutination separation unit 130. Consequently, a multi-layered blood of plasma and blood cells can be automatically formed in the blood-cell agglutination separation unit 130.

Referring back to FIG. 6, in another preferred embodiment, the blood-cell agglutination separation unit 130 includes a first discharge channel and a second discharge channel, as indicated by arrows in FIG. 6, corresponding to the blood-cell agglutination area 13a and the platelet-rich plasma area 13b. In a blood cell separation operation, the first discharge channel can supply a blood-cell-rich blood and the second discharge channel can supply a platelet-rich plasmatic solution.

Still referring to FIG. 6, in another preferred embodiment, after discharging the blood-cell-rich blood and the platelet-rich plasmatic solution to predetermined separate blood containers or container-like devices, another predetermined amount of (new) untreated blood 3 is further supplied to the blood-cell agglutination separation unit 130 for continuously and effectively separating blood cells from platelets and plasmas.

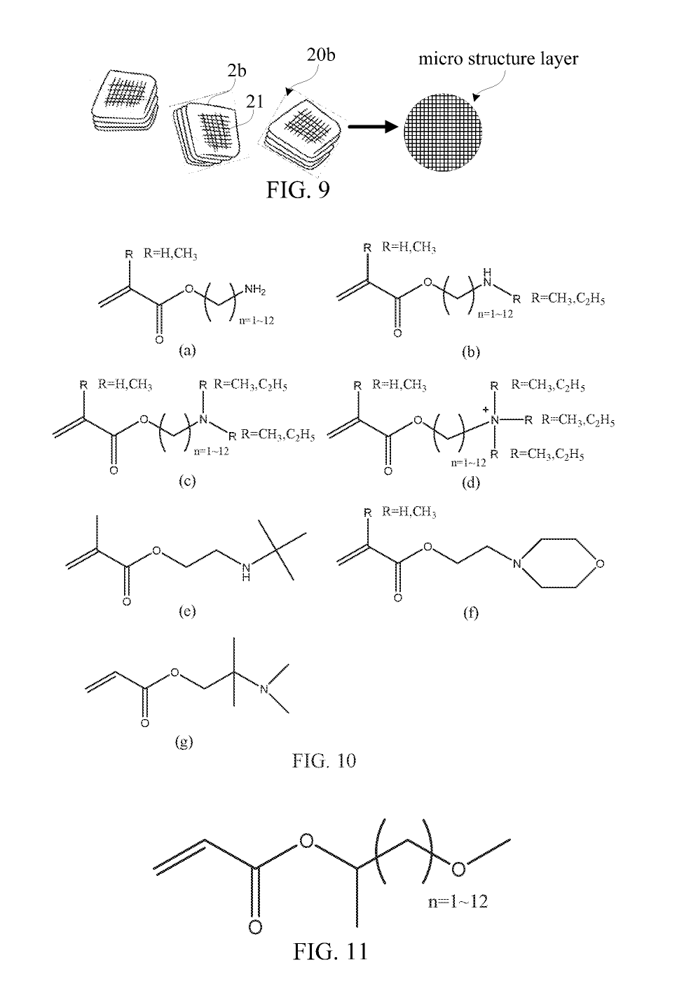

Still referring to FIG. 6, by way of example, the blood-cell agglutination separation material 20 has an additive material selected from blood-cell agglutination gel or blood-cell agglutination paste. A porous material, a micro structure material or a micro structure layer is coated by or immersed in the blood-cell agglutination gel or blood-cell agglutination paste, as best shown in FIGS. 8 and 9.

FIG. 8 shows a schematic block diagram of a blood-cell agglutination separation device in accordance with a fifth preferred embodiment of the present invention. Referring now to FIG. 8, in comparison with the fourth preferred embodiment, the blood-cell agglutination separation device in accordance with the fifth preferred embodiment of the present invention includes a blood-cell agglutination separation material 20a having a blood-cell separation substrate or unit 2a which is formed from a bandage roll material with a number of micro structure layer areas or films to receive the blood-component adhesion material 21. The blood-component adhesion material 21 is adhered to opposite bandage surfaces of the blood-cell separation substrate or unit 2a with the micro structure layer areas.

FIG. 9 shows a schematic block diagram of a blood-cell agglutination separation device in accordance with a sixth preferred embodiment of the present invention. Referring now to FIG. 9, in comparison with the fourth preferred embodiment, the blood-cell agglutination separation device in accordance with the sixth preferred embodiment of the present invention includes a blood-cell agglutination separation material 20b having a blood-cell separation substrate or unit 2b which is formed from a gauze pad material with a number of micro structure layer areas or films to receive the blood-component adhesion material 21. The blood-component adhesion material 21 is disposed on a central area of the blood-cell separation substrate or unit 2b with the micro structure layer areas.

FIGS. 10(a)-10(g) show a set of chemical structure views of a series of amino monomers applied in a blood-component adhesion material in accordance with a preferred embodiment of the present invention. Referring now to FIGS. 8, 9 and 10(a) to 10(g), by way of example, the blood-component adhesion material 21 includes several types of amine monomer materials (i.e. primary amines, secondary amines, tertiary amines, a positively charged monomer or a combination thereof) and other suitable materials.

FIG. 11 shows a chemical structure view of monomer having a hydrophobic radical group applied in the blood-component adhesion material in accordance with another preferred embodiment of the present invention. Referring now to FIGS. 8, 9 and 11, a monomer material is selected from an ether acrylic monomer having a long carbon chain suitable for manufacturing the blood-component adhesion material 21. A carbon number of the carbon chain of the blood-component adhesion material 21 is preferably ranging from 1 to 12. Furthermore, the blood-component adhesion material 21 is made of dipropylene glycol methyl ether diacrylate, propylene glycol methyl ether acrylate and isomers thereof.

Although the invention has been described in detail with reference to its presently preferred embodiments, it will be understood by one of ordinary skills in the art that various modifications can be made without departing from the spirit and the scope of the invention, as set forth in the appended claims.

What is claimed is:

1. A blood-cell agglutination separation method comprising:
prefabricating a blood-cell separation substrate or unit;
prefabricating a blood-component adhesion material which is made of at least one monomer material;
providing a predetermined amount of the blood-component adhesion material on the blood-cell separation substrate or unit to form a blood-cell agglutination separation material; and
in blood cell separation, the blood-cell agglutination separation material attracting blood platelets and functional groups of a predetermined amount of blood proteins to thereby cause unbalance of blood for automatically accelerating precipitation and isolation of blood corpuscles from blood plasma.

2. The blood-cell agglutination separation method as defined in claim 1, wherein a predetermined amount of blood is supplied by a blood supply unit or an injector.

3. The blood-cell agglutination separation method as defined in claim 1, wherein the blood-cell separation substrate or unit is made of a cotton material, a gauze material, a bandage material or a combination thereof.

4. The blood-cell agglutination separation method as defined in claim 1, further separately supplying a blood-cell-rich blood via a first discharge channel of a blood-cell agglutination separation unit and a platelet-rich plasmatic solution via a second discharge channel of the blood-cell agglutination separation unit.

5. The blood-cell agglutination separation method as defined in claim 1, wherein a substrate further includes a blood cell adsorbent, a blood cell adsorption material or a blood cell adsorption gel and is provided in a blood-cell agglutination separation unit.

6. A blood-cell agglutination separation method comprising:
providing a predetermined amount of blood-component adhesion material on a blood-cell separation substrate or unit to form a blood-cell agglutination separation material in a blood-cell agglutination separation unit;
supplying a predetermined amount of blood into the blood-cell agglutination separation unit; and
utilizing the blood-cell agglutination separation material to attract blood platelets and functional groups of a predetermined amount of blood proteins to thereby cause unbalance of the predetermined amount of blood for automatically accelerating precipitation and isolation of blood corpuscles from blood plasma.

7. The blood-cell agglutination separation method as defined in claim 6, wherein the predetermined amount of blood is supplied by a blood supply unit or an injector.

8. The blood-cell agglutination separation method as defined in claim 6, wherein the blood-cell separation substrate or unit is made of a cotton material, a gauze material, a bandage material or a combination thereof.

9. The blood-cell agglutination separation method as defined in claim 6, further separately supplying a blood-cell-rich blood via a first discharge channel of the blood-cell agglutination separation unit and a platelet-rich plasmatic solution via a second discharge channel of the blood-cell agglutination separation unit.

10. The blood-cell agglutination separation method as defined in claim 6, wherein a substrate further includes a blood cell adsorbent, a blood cell adsorption material or a blood cell adsorption gel and is provided in the blood-cell agglutination separation unit.

11. The blood-cell agglutination separation method as defined in claim 6, wherein the blood-cell separation substrate or unit includes a micro structure layer or film to receive the blood-component adhesion material.

12. A blood-cell agglutination separation device comprising:
a blood-cell agglutination separation unit to contain a predetermined amount of blood, with the blood-cell agglutination separation unit having a blood-cell agglutination area and a platelet-rich plasma area for an operation of blood-cell agglutination separation;
a blood-cell separation substrate or unit provided in the blood-cell agglutination separation unit; and
a blood-cell agglutination separation material formed from a predetermined amount of the blood-component adhesion material provided on the blood-cell separation substrate or unit, with the blood-component adhesion material is made of at least one monomer material;
wherein in blood cell separation, the blood-cell agglutination separation material attracts blood platelets and functional groups of a predetermined amount of blood proteins to thereby cause unbalance of the predetermined amount of blood for automatically accelerating precipitation and isolation of blood corpuscles from blood plasma.

13. The blood-cell agglutination separation device as defined in claim 12, wherein the blood-cell agglutination separation unit is formed from a separation bottle device, a blood-passage separation bottle device or a negative pressure vessel device.

14. The blood-cell agglutination separation device as defined in claim 12, wherein the predetermined amount of blood is supplied to the blood-cell agglutination separation unit by a blood supply unit or an injector.

15. The blood-cell agglutination separation device as defined in claim 12, wherein the blood-cell separation substrate or unit is made of a cotton material, a gauze material, a bandage material or a combination thereof.

16. The blood-cell agglutination separation device as defined in claim 12, wherein the blood-cell agglutination separation unit includes a first discharge channel for supplying a blood-cell-rich blood and a second discharge channel for supplying a platelet-rich plasmatic solution.
